(19) United States
(12) Patent Application Publication
(10) Pub. No.: US 2008/0269899 A1
(43) Pub. Date: Oct. 30, 2008

(54) ALLOGRAFT ANTERIOR CERVICAL PLATING SYSTEM

(76) Inventor: Kenneth L. Horton, Birmingham, AL (US)

Correspondence Address: BUSH INTELLECTUAL PROPERTY LAW GROUP, LLC, P.O. BOX 381146, BIRMINGHAM, AL, 35238, US

(21) Appl. No.: 11/831,635

(22) Filed: Jul. 31, 2007

Related U.S. Application Data
(60) Provisional application No. 60/914,522, filed on Apr. 27, 2007.

(51) Int. Cl. A61F 5/00; A61B 17/56; A61B 17/58; A61B 19/00; A61B 17/32; A61B 17/70; A61B 17/04; A61F 2/44

(52) U.S. Cl. 623/17.15; 606/80; 606/99; 606/96; 128/898; 606/301; 606/280; 606/184

(57) ABSTRACT

A method for inserting an allograft implant between adjacent cervical vertebrae to fuse the vertebrae together and a surgical kit for use therein. The kit preferably comprises an allograft implant, a plurality of bio-absorbable screws, a forceps-type implant inserter tool, a drill, a drill guide, a tapping tool, and a screwdriver. The method preferably comprises the steps of preparing a surface of a vertebra to receive an allograft plate implant, placing the implant against the surface of the vertebra, placing a drill guide against an exposed surface of the implant, inserting a drill though the drill guide and drilling a plurality of holes through the implant and into the vertebra to a predetermined depth, tapping the plurality of holes through the implant and into the vertebra to create screw threads, securing the implant to the vertebra by inserting a bio-absorbable screw into each of the plurality of holes through the implant and into the vertebra, and removing any excess portion of each screw outside of the plurality of holes.

### REFERENCE TO RELATED PATENT APPLICATION

The present application claims priority to U.S. Provisional Application No. 60/914,522, filed on Apr. 27, 2007, the disclosure of which is incorporated herein by reference.

### FIELD OF THE INVENTION

The present invention relates to surgical implant systems, and more particularly, to allograft implant methods used to fuse cervical vertebrae and surgical kits therefor.

### BACKGROUND OF THE INVENTION

The neck is the upper portion of the spine and is made up of the seven upper vertebrae which are often referred to as the cervical spine. Numerous cervical spine disorders require surgery for relief of painful symptoms. One of the basic underlying factors associated with most spine disorders is the dehydration of the disks. Herniated cervical disk is a common neck pain diagnosis which results when the center of the nucleus bulges through the annulus and presses on a nerve, resulting in neck or arm pain, or weakness in the arm. Cervical fusion involves the stabilization of two or more vertebrae by locking them together. One can approach the cervical spine through a small incision in the front of the neck, usually within a skin fold line under the chin. After retracting neck muscles, the affected intervertebral disk is removed. This is called decompression. After removal, a bone graft may be inserted into the intervertebral region to fuse the upper and lower vertebrae together. However, mechanical fusion of the adjoining vertebrae may also be required.

Anterior cervical plating systems are used for mechanical fusion of the adjoining vertebrae and are well known. All currently available plating systems use a metal plate, usually titanium, screws that go through the plate into the vertebra, and a locking mechanism whereby the screw is locked to the metal plate. Metal plate designs are rigid and prevent loads from being transmitted through bone grafts, which can interfere with fusion and allow for grafts to be reabsorbed. The appearance of metal implants on x-rays tends to have an artificial fuzziness, which makes assessment of fusion, one of the clinical criteria of a successful interbody fusion device, difficult. Moreover, metals tend to have mechanical properties that are unevenly matched to bone. The metal fusion or plating device can break, the screws can back out, and the metal can fatigue. Metal plates often cause pain, requiring further surgery. Metal plating is too rigid by design, not allowing bone to go through normal healing, which can cause bone resorption. Ideal plating needs to be dynamic, allowing microflexion and movement.

What is needed, and is not found in the prior art, is an allograft cervical plating system that overcomes the disadvantages of metal plating systems.

### SUMMARY OF THE INVENTION

The present invention comprises a surgical kit for use in a method for inserting an allograft implant between adjacent cervical vertebrae to fuse the vertebrae together, preferably comprising an allograft implant, a plurality of bio-absorbable screws, a forceps-type implant inserter tool, a drill, a drill guide, a tapping tool, and a screwdriver. The invention further comprises a method for inserting an allograft implant between adjacent cervical vertebrae to fuse the vertebrae together, preferably comprising the steps of removing an intervertebral disc from between the adjacent vertebrae, inserting a spacer between the adjacent vertebrae, preparing a surface of a vertebra to receive an implant, placing the allograft plate implant against the surface of the vertebra, placing a drill guide against an exposed surface of the allograft plate, inserting a drill though the drill guide and drilling a plurality of holes through the allograft plate and into the vertebra to a predetermined depth, tapping the plurality of holes through the allograft plate and into the vertebra to create screw threads, securing the allograft plate to the vertebra by inserting a bio-absorbable screw into each of the plurality of holes through the allograft plate and into the vertebra, removing any excess portion of each screw outside of the plurality of holes, and removing the spacer from between the adjacent vertebra.

These and other features of the invention will become apparent from the following detailed description of the preferred embodiments of the invention.

### BRIEF DESCRIPTION OF THE DRAWINGS

FIG. 1 shows the principal surgical kit components of the present invention.

FIG. 2 is an enlarged view of the tips of alternate embodiments of the implant inserter tool.

FIG. 3 shows the allograft implant of the present invention.

### DETAILED DESCRIPTION OF THE PREFERRED EMBODIMENTS

The system of the present invention for cervical fusion comprises an allograft implant preferably harvested from human cortical bone as a bone plate with bioabsorbable or metal screws. The system provides stabilization for vertebral bodies that achieves fusion of the vertebrae. The system controls vertebral motion for a period of time to allow for the fusion. The allograft will fuse to the vertebrae because the cortical bone is biologically inert. Fusion happens because the vertebral body is decorticated, causing bleeding, which stimulates a healing process in the vertebra. The decortication, in effect, creates a natural “fracture” with the tendency to heal. The allograft is strong because it is derived from femur and tibia bones, and mimics the elastic properties of bone because it is bone, unlike currently used metal fusion plates. The system has both single level and multilevel applications.

In the multilevel applications, the allografts are attached in an “infinity” (non-contiguous) design. The strength and rigidity of cortical bone are well established. Threaded bioabsorbable screws are preferably used to attach the allograft plate to the vertebrae. Hot loop cautery is used to cut the bioabsorbable screws, resulting in a melting down or waxing technique, which seals the head of the screws. This system and method does not preclude other surgery.

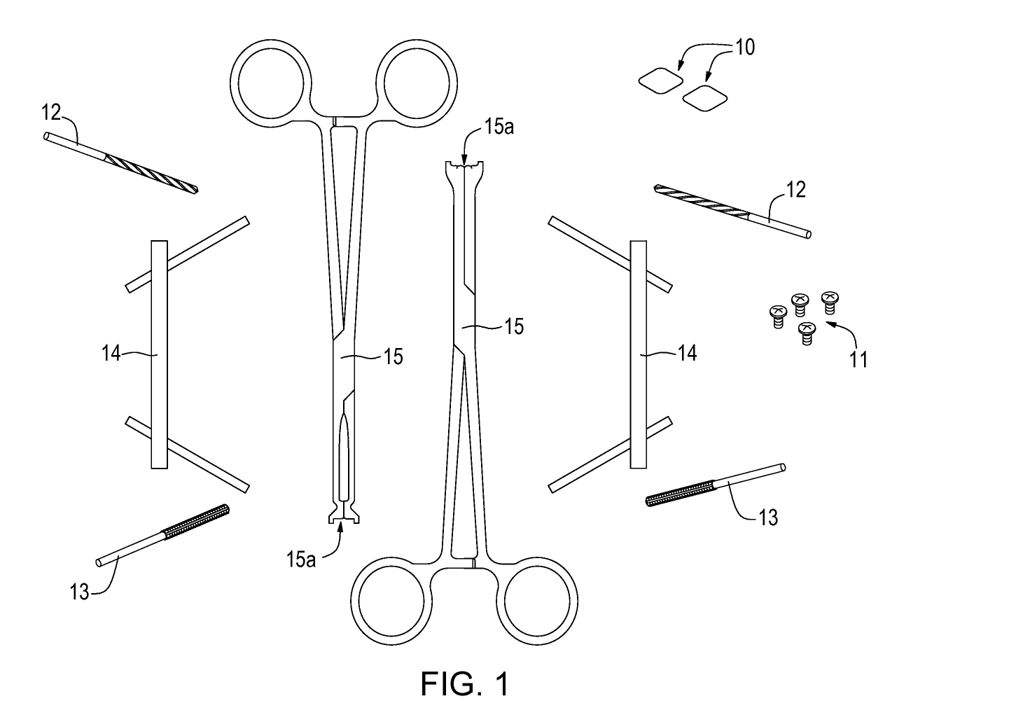

Several devices are used in the system of the present invention, the principals of which are illustrated in FIG. 1. A bone allograft plate 10 is used as a plate to join two adjacent vertebrae. The allograft plate, best shown in FIG. 3, is preferably slightly convex with a medial/lateral curvature, rectangular, tapered, and has rounded corners. The thickness of the allograft plate is at least 2 mm, and the plate may be pre-drilled. Bio-absorbable screws 11 are preferably made of poly-lactic-lactic-acid (PLLA), which has a proven strength characteristic, and are threaded to prevent backout. These PLLA screws are resorbed by a process of hydrolysis over a period between 12-24 months. The long resorption time helps maintain stabilization of the plate. Screws maintain over 90% of structural strength through 12 weeks. Eventually, as the screws resorb, the allograft plate fuses and becomes 100% bone, with the allograft being replaced by the patient's own bone through a process known as creeping substitution.

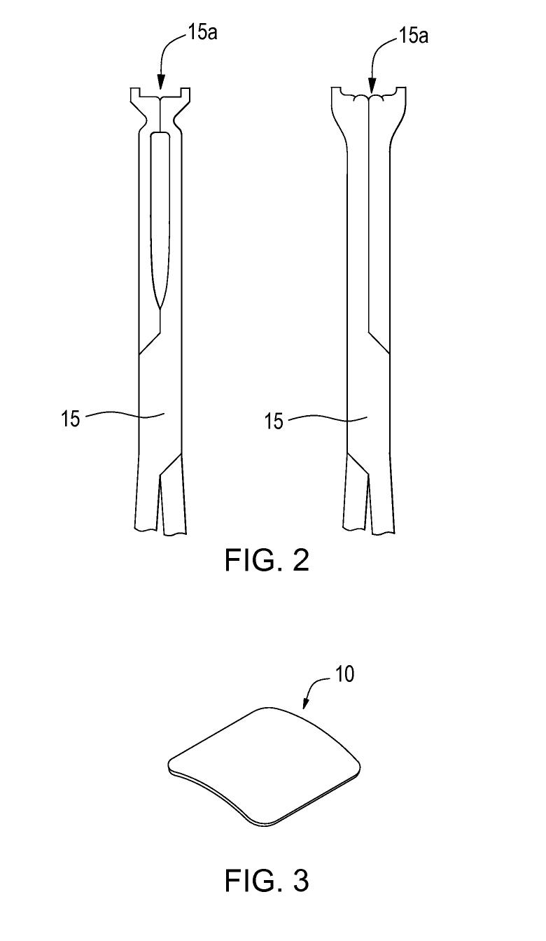

Drills 12 are used to drill holes in the bone plate 10 and vertebra. Taps 13 are used to thread the holes to accommodate the bioabsorbable screws 11. Plate and bone holder pins may be used in opposing corners of the plate to allow for easier drilling of the bioabsorbable screws. The pins are threaded with a stop. Drill and tap guides or sleeves 14 are used to accurately place the drills and taps. An inserter tool or plate holder 15 is preferably forceps-like and is used to grasp and insert the allograft plate into position on the anterior aspect of the vertebrae. A screwdriver is used to operate the taps and to insert the bioabsorbable screws. FIG. 2 shows an enlarged view of the grasping tips 15a of 2 embodiments of the inserter tool.

According to the surgical method of the present invention, vertebral levels to be fused are first prepared in the usual manner with discectomy and abrasion. A spacer is then put between vertebral bodies that are going to be fused, after which, the anterior surfaces of the vertebral bodies are prepared according to the surgeon's judgment or preference. The allograft bone plate 10 is rehydrated, loaded into the inserter tool 15 and held onto the two vertebral bodies for placement trial and for screw placement orientation. The bone holding pins are placed through the plate into the vertebral bodies above and below at opposing corners. Holes in the plate are prepared by drilling through the plate into the vertebral body to a pre-determined depth specific for the patient's anatomy. Drill guide 14 is used to regulate depth and angle of the hole. Drill holes are tapped using appropriately sized tap 13. Bio-absorbable screws 11 are placed into the plate and through the vertebral body. Excess screw above the plate is severed with hot loop cautery. Hot loop cautery results in melted material that seals the top of the screw providing a bridge between the screw and plate interface. At least 4 bio-absorbable screws are used for each allograft plate. This system could be used for single or multi-level fusions.

Some or all of the following devices can be used to form a surgical kit for allograft anterior cervical vertebral fusion: an allograft plate 10, bio-absorbable screws 11, drills 12, taps 13, holder pins (not shown), drill and tap guides 14, forceps-like plate holders 15, and a screwdriver (not shown).

While the invention has been shown and described in some detail with reference to specific exemplary embodiments, there is no intention that the invention be limited to such detail. On the contrary, the invention is intended to include any alternative or equivalent embodiments that fall within the spirit and scope of the invention as described herein and as recited in the appended claims.

1. A method for inserting an allograft implant between adjacent cervical vertebrae to fuse the vertebrae together, comprising the steps of:
a) preparing a surface of a vertebra to receive an implant;
b) placing said implant against the surface of the vertebrae of step a, wherein said implant is an allograft plate;
c) placing a drill guide against an exposed surface of said allograft plate;
d) inserting the drill though said drill guide and drilling a hole through said allograft plate and into the vertebra of step a to a predetermined depth;
e) tapping said hole through said allograft plate and into the vertebra of step a to create screw threads; and
f) securing said allograft plate to the vertebra of step a by inserting a bioabsorbable screw into said hole through said allograft plate and into the vertebra of step a.

2. The method according to claim 1, wherein steps c and d are repeated to obtain a plurality of holes through said allograft plate and into the vertebra of step a.

3. The method according to claim 2, wherein step e further comprises tapping said plurality of holes through said allograft plate and into the vertebra of step a to create screw threads.

4. The method according to claim 3, wherein step f further comprises securing said allograft plate to the vertebra of step a by inserting a bioabsorbable screw into each of said plurality of holes through said allograft plate and into the vertebra of step a.

5. The method according to claim 4, further comprising the step of removing any excess portion of each said screw outside of said plurality of holes after step f.

6. The method according to claim 5, wherein said excess portion of each said screw is removed by hot loop cautery, thereby sealing each said screw to said allograft plate.

7. A method for inserting an allograft implant between adjacent cervical vertebrae to fuse the vertebrae together, comprising the steps of:
a) removing an intervertebral disc from between the adjacent vertebrae;
b) inserting a spacer between the adjacent vertebrae;
c) preparing a surface of a vertebra to receive an implant;
d) placing said implant against the surface of the vertebra of step c, wherein said implant is an allograft plate;
e) placing a drill guide against an exposed surface of said allograft plate;
f) inserting a drill though said drill guide and drilling a hole through said allograft plate and into the vertebra of step e to a predetermined depth;
g) repeating steps e and f to obtain a plurality of holes through said allograft plate and into the vertebra of step c;
b) tapping said plurality of holes through said allograft plate and into the vertebra of step c to create screw threads;
i) securing said allograft plate to the vertebra of step c by inserting a bioabsorbable screw into each of said plurality of holes through said allograft plate and into the vertebra of step c;
j) removing any excess portion of each said screw outside of said plurality of holes; and
k) removing said spacer from between the adjacent vertebrae.

8. The method according to claim 7, wherein said allograft plate is placed against the surface of the vertebra in step d with a plate inserter tool.

9. The method according to claim 7, wherein said excess portion of each said screw is removed in step j by hot loop cautery, thereby sealing each said screw to said allograft plate.

10. A surgical kit for use in a method for inserting an allograft implant between adjacent cervical vertebrae to fuse the vertebrae together, comprising:
a) at least one allograft implant;
b) at least one bioabsorbable screw;
c) an implant inserter tool;
d) a drill;
e) a drill guide;
f) a tapping tool; and
g) a screwdriver.

11. A surgical kit according to claim 10, wherein said at least one allograft implant comprises a rectangular plate having rounded corners, a medial to lateral curvature, and a thickness tapering from a central portion of said implant towards edges of said implant.

12. A surgical kit according to claim 10, wherein said at least one bio-absorbable screw comprises a poly-lactic-lactic-acid material.

13. A surgical kit for use in a method for inserting an allograft implant between adjacent cervical vertebrae to fuse the vertebrae together, comprising:
a) an allograft implant;
b) a plurality of bio-absorbable screws;
c) a forceps-type implant inserter tool;
d) a drill;
e) a drill guide;
f) a tapping tool; and
g) a screwdriver.

14. A surgical kit according to claim 13, wherein said allograft implant comprises a rectangular plate having rounded corners, a medial to lateral curvature, and a thickness tapering from a central portion of said implant towards edges of said implant.

15. A surgical kit according to claim 13, wherein said bio-absorbable screws comprise a poly-lactic-lactic-acid material.
